(19) United States
(12) Patent Application Publication
(10) Pub. No.: US 2008/0108681 A1
(43) Pub. Date: May 8, 2008

(54) Use of allantoin as a pro-collagen synthesis agent in cosmetic compositions

(75) Inventors: John V. Scimeca, Kentwood, MI (US); David J. Fast, Grand Rapids, MI (US); Amy C. Zimmerman, Grand Rapids, MI (US)

Correspondence Address: IN RE: ALTICOR INC. 28533;BRINKS, HOFER, GILSON & LIONE, P.O. BOX 10395, CHICAGO, IL, 60610, US

(73) Assignee: Access Business Group International LLC

(21) Appl. No.: 11/977,926

(22) Filed: Oct. 26, 2007

Related U.S. Application Data
(60) Provisional application No. 60/855,102, filed on Oct. 27, 2006.

(51) Int. Cl. A61K 31/4164; A61P 17/00; A61Q 19/08

(52) U.S. Cl. 514/390

(57) ABSTRACT

Compositions comprising allantoin and an acceptable carrier and methods of using such compositions to increase pro-collagen synthesis in skin are disclosed. Compositions of the present invention may be used to decrease the signs of skin aging such as wrinkles and fine lines. Compositions of the present invention may be topically administered, orally administered or parenterally, such as administration by injection. When topically administered, additive ingredients such as penetration enhancers, fragrances, and moisturizers and cosmetic adjuvants may be included in compositions of the present invention.

This application claims priority to U.S. Provisional Patent Application Ser. No. 60/855,102, filed Oct. 27, 2006, the entire contents of which are hereby incorporated by reference.

### BACKGROUND

Collagen is the primary protein of connective tissue, which includes cartilage, bone, tendon, teeth, and skin. Collagen, in a pre-processed form called pro-collagen, is assembled in cells and consists of three polypeptides wound around each other in a triple helix form, which is stabilized by intrachain disulfide bonds. After the helical molecule is assembled and modified in the cell it is secreted into the extracellular medium and further processed to a mature form known as tropocollagen.

In one example, collagen, specifically type I collagen, may be formed as follows: Three peptide chains are formed (2 alpha-1 chains and 1 alpha-2 chain) in ribosomes along the rough endoplasmic reticulum (RER). These peptide chains (known as prepro-collagen) have a registration and a signal peptide attached to each end. The peptide chains are sent into the lumen of the RER where the signal peptides are cleaved to form pro-collagen. Hydroxylation of lysine and proline amino acids in the pro-collagen occurs inside the lumen. This process is dependent on Ascorbic Acid (Vitamin C) as a cofactor. Glycosylation of specific hydroxylated amino acids also occurs in the lumen leading to formation of a triple helical pro-collagen structure.

The pro-collagen is then shipped to the golgi apparatus, where it is packaged and secreted by exocytosis. Outside the cell, registration peptides are cleaved and tropocollagen is formed by pro-collagen peptidase. Multiple tropocollagen molecules form collagen fibrils, and multiple collagen fibrils form into collagen fibers.

Matured collagen molecules assemble into fibrils in the extracellular space in a staggered, parallel, fashion wherein the molecules are stabilized in this fibril pattern by covalent cross-linking bonds between the N-terminus of one molecule and the C-terminus of another. The collagen fibrils are interlaced and branched in skin. These interlaced, branched collagen fibrils provide the skin with its shape and firmness, while another skin protein, elastin, provides skin with its elasticity. Elastin coils and recoils like a spring and accounts for the elasticity of structures such the skin, blood vessels, heart, lungs, intestines, tendons, and ligaments. Elastin is normally not produced by the human body after puberty and aging begin.

Besides its many structural properties, collagen serves as the major catalyst for growth and repair of nearly all the body's tissues. For example, fibroblasts, the primary cell type in the dermis, require interaction with collagen fibrils in order to maintain normal function. Many different aging diseases are related to the body's supply of this vital protein and to disorders in the collagen itself. In fact, the body's production of collagen slows dramatically with aging. In addition, like all other proteins in the human body, collagen and elastin are constantly being degraded and lost. Degradation of collagen in the skin can cause a loss of smoothness, evenness, firmness and lead to sagging and the formation of wrinkles.

Collagen has many uses. For example, collagen is included in many skin treatments, including skin moisturizers, and may also be used to reduce scar formation in cosmetic surgery and in treatment of burns. However, difficulties have been encountered in humans being able to effectively and efficiently utilize collagen obtained from supplements. Therefore, a composition capable of combating loss of collagen by increasing a body's own collagen production and/or production of precursors of collagen, such as pro-collagen, would be useful for improving the appearance, texture, and firmness of the skin and maintaining general skin health.

### BRIEF SUMMARY

Loss of collagen is directly related to numerous diseases and disorders including skin aging. Indeed, loss of collagen is a major contributor to the formation wrinkles. The present invention is based on the surprising discovery that administration of a composition comprising allantoin stimulates and increases synthesis of pro-collagen, a precursor to collagen. Thus, in one example, the present invention is a composition for stimulating pro-collagen and/or collagen synthesis, wherein the composition comprises allantoin and an acceptable carrier.

In one example, allantoin is present in a composition of the present invention in an amount ranging from approximately 0.05% w/w to approximately 2% w/w of the composition. In another example, a composition of the present invention comprises allantoin ranging from approximately 0.075% w/w to approximately 1.5% w/w of the composition. In a further example, a composition of the present invention comprises allantoin ranging from approximately 0.1% w/w to approximately 1% w/w of the composition. In another example, a composition of the present invention comprises allantoin ranging from approximately 0.15% w/w to approximately 0.75% w/w of the composition. In a further example, a composition of the present invention comprises allantoin in an amount of approximately 0.5% w/w of the composition.

In another example, the present invention is a method of increasing pro-collagen and/or collagen synthesis in mammalian skin comprising administering a composition comprising allantoin. In a further example, the mammalian skin is human skin. In yet a further example, the composition is topically administered. In other examples, the composition may be orally administered or parenterally administered, such as administered by injection.

In a further example, the present invention is a method of improving the appearance, texture, and firmness of mammalian skin comprising administering a composition comprising allantoin, wherein the composition increases synthesis of pro-collagen and/or collagen in the skin. In one example, the composition is topically administered to human skin. In other examples, the composition may be orally administered or parenterally administered, such as administered by injection.

In another example, the present invention is a method of decreasing the appearance and/or depth of wrinkles or fine lines in mammalian skin comprising administering a composition comprising allantoin, wherein the composition increases synthesis of pro-collagen and/or collagen in the skin.

### DETAILED DESCRIPTION

It is to be understood that this invention is not limited to the particular compositions, methodology, or protocols described herein. Further, unless defined otherwise, all technical and scientific terms used herein have the same meaning as commonly understood to one of ordinary skill in the art to which this invention belongs. It is also to be understood that the terminology used herein is for the purpose of describing particular examples only, and is not intended to limit the scope of the present invention, which will be limited only by the claims.

The present invention is based on the surprising discovery that allantoin (C₄H₆N₄O₃) (2,5-dioxy-4-imidazolidinyl urea) stimulates pro-collagen synthesis in human skin. An increase in pro-collagen synthesis in turn leads to an increase in collagen synthesis. Therefore, the present invention also is based on the surprising discovery that allantoin stimulates collagen synthesis in human skin.

Allantoin also is known as 5-ureidohydantoin, glyoxyldiureide, 5-ureidohydantoin, udder cream, ureidohydantoin, glyoxyldiureide, and hemocane. It is a product of oxidation of uric acid. Allantoin generally is obtained as a botanical extract of the comfrey plant but also may be obtained from other sources. Allantoin helps to heal wounds and skin irritations and stimulate growth of healthy tissue.

Allantoin is used in numerous products ranging from anti-acne products, sun care products, and clarifying lotions because of its ability to help heal minor wounds and promote healthy skin to skin softening cosmetic preparations because of its keratolytic effect and abrasive and astringent properties. Specifically, allantoin acts as chemical debrider of necrotic and scaling tissue, cleansing the areas where applied. Allantoin also frequently is present in toothpaste, mouthwash, and other oral hygiene products, in shampoos, lipsticks, various cosmetic lotions and creams, and other cosmetic and pharmaceutical products. However, until now it was not known that allantoin could be used in these products as a means for increasing pro-collagen and/or collagen synthesis and/or production.

Allantoin has limited solubility; typically at least 190 ml of water are required to solubilize 1 gram of allantoin. Therefore, in compositions of the present invention, allantoin is present at relatively low concentrations, for example ranging from approximately 0.05% w/w to approximately 2% w/w of the composition. In one example, a composition of the present invention comprises allantoin ranging from approximately 0.075% w/w to approximately 1.5% w/w of the composition. In a further example, a composition of the present invention comprises allantoin ranging from approximately 0.1% w/w to approximately 1% w/w of the composition. In another example, a composition of the present invention comprises allantoin ranging from approximately 0.15% w/w to approximately 0.75% w/w of the composition. In a further example, a composition of the present invention comprises allantoin in an amount of approximately 0.5% w/w of the composition.

The compositions of the present invention may be administered with an acceptable carrier, additives, and/or cosmetic adjuvants. For example, a composition of the present invention could be externally administered with an acceptable carrier in the form of a gel, lotion, cream, tonic, emulsion, etc. As a further example, a composition of the present invention could be internally administered with an acceptable carrier in the form of a pill, tablet, powder, bar, beverage, etc. Thus, the compositions described herein are useful in a wide variety of finished products, including cosmetic products, pharmaceutical products, food products, and beverage compositions. Preferably, the products are useful for increasing the synthesis of pro-collagen and/or collagen in skin thereby providing the skin with an improved texture and appearance.

In another embodiment of the invention, the compositions of the present invention are topically administered in the form of a: solution, gel, lotion, cream, ointment, oil-in-water emulsion, water-in-oil emulsion, stick, spray, paste, mousse, tonic, or other cosmetically and topically suitable form.

Preferably, compositions of the present invention that are suitable for topical administration are mixed with an acceptable carrier. An acceptable carrier may act variously as solvent, carrier, diluent or dispersant for the constituents of the composition, and allows for the uniform application of the constituents to the surface of the skin at an appropriate concentration. The acceptable carrier may also facilitate penetration of the composition into the skin.

In one example of a composition of the present invention for topical application, the acceptable carrier forms from about 80% to about 99.99% by weight of the total composition. In other examples, the acceptable carrier will form from about 90% to 99% by weight of the total composition. The acceptable carrier may also form from about 91% to about 98% by weight of the total composition; from about 92% to about 97% by weight of the total composition; from about 93% to about 96% by weight of the total composition; or from about 94% to about 95% by weight of the total composition. The acceptable carrier can, in the absence of other cosmetic adjuncts or additives, form the balance of the composition.

The allantoin and other ingredients used in practicing the present invention may be soluble or insoluble in the acceptable carrier. If all ingredients of a composition are soluble in the acceptable carrier, then the vehicle acts as solvent. However, if all or some ingredients of a composition are insoluble in the acceptable carrier, then those ingredients are dispersed in the vehicle by means of, for example, a suspension, emulsion, gel, cream or paste, and the like.

Thus, it will be apparent to the skilled artisan that the range of possible acceptable carriers is very broad. For example, acceptable carriers can be emulsions, lotions, creams, or tonics. Acceptable carriers can comprise water, ethanol, butylene glycol, or other various solvents that aid in penetration of the skin. Some examples of suitable vehicles are described in U.S. Pat. No. 6,184,247 and in U.S. Pat. No. 6,579,516, the entire contents of which are incorporated herein by reference.

In general, acceptable carriers according to the present invention may comprise, but are not limited to comprising, any of the following examples: water; castor oil; ethylene glycol monobutyl ether; diethylene glycol monoethyl ether; corn oil; dimethyl sulfoxide; ethylene glycol; isopropanol; soybean oil; glycerin; soluble collagen; zinc oxide; titanium oxide; or Kaolin.

In one aspect, the acceptable carrier used in practicing the present invention comprises water and ethanol. Optionally, the acceptable carrier also contains butylene glycol and/or frescolate MGA. For example, the acceptable carrier can comprise 40-60% water, 45-55% ethanol, and 5-10%% butylene glycol by weight of the composition. In practicing the present invention, preferably this acceptable carrier is mixed with allantoin comprising from approximately 0.05% to approximately 2% by weight of the total composition.

Additionally, acceptable carriers used in the present invention may optionally comprise one or more humectants, including but not limited to: glycerin, dibutyl phthalate; soluble collagen; sorbitol; or sodium 2-pyrrolidone-5-carboxylate. Other examples of humectants that may be used in practicing the present invention can be found in the CTFA (Cosmetic Toiletry and Fragrance Association) Cosmetic Ingredient Handbook, the relevant portions of which are incorporated herein by reference.

Additionally, acceptable carriers in the present invention may optionally comprise one or more emollients including but not limited to: isononyl isononanoate, butane-1,3-diol; cetyl palmitate; dimethylpolysiloxane; glyceryl monoricinoleate; glyceryl monostearate; isobutyl palmitate; isocetyl stearate; isopropyl palmitate; isopropyl stearate; butyl stearate; isopropyl laurate; hexyl laurate; decyl oleate; isopropyl myristate; lauryl lactate; octadecan-2-ol; caprylic triglyceride; capric triglyceride; polyethylene glycol; propane-1,2-diol; triethylene glycol; sesame oil; coconut oil; safflower oil; isoamyl laurate; nonoxynol-9; panthenol; hydrogenated vegetable oil; tocopheryl acetate; tocopheryl linoleate; propylene glycol; arachis oil; castor oil; isostearic acid; palmitic acid; isopropyl linoleate; lauryl lactate; myristyl lactate; decyl oleate; or myristyl myristate. Other examples of emollients that may be used in practicing the present invention can be found in the CTFA Cosmetic Ingredient Handbook, the relevant portions of which are incorporated herein by reference.

Additionally, acceptable carriers used in the present invention may optionally comprise one or more penetration enhancers including but not limited to: pyrrolidones, for example 2-pyrrolidone; alcohols, such as ethanol; alkanols, such as decanol; glycols, such as propylene glycol, dipropylene glycol, butylenes glycol; surfactants; or terpenes.

Other acceptable carriers that may be used in practicing the present invention will be apparent to those of skill in the art and are included within the scope of the present invention.

For example, an acceptable carrier can be a lotion that is topically applied. The lotion may comprise, in addition to allantoin, carbomer 981, water, glycerin, isopropyl myristate, mineral oil, shea butter, stearic acid, glycol stearate, cetyl alcohol, dimethicone, preservatives, and tea. In another example, the lotion may comprise, in addition to allantoin, water, isononyl isononanoate, hydrogenated lecithin, butylene glycol, glycerin, caprylic/capric triglyceride, stearic acid, cetyl alcohol, behenyl alcohol, xanthan gum, methylparaben, and chlorphenesin.

In another example, the compositions of the present invention are administered orally in the form of a liquid or a solid. The liquid may be water-based, milk-based, tea-based, fruit juice-based, or some combination thereof. Solid and liquid compositions for internal administration according to the present invention can further comprise thickeners, including xanthum gum, carboxymethyl-cellulose, carboxyethylcellulose, hydroxypropylcellulose, methylcellulose, microcrystalline cellulose, starches, dextrins, fermented whey, tofu, maltodextrins, polyols, including sugar alcohols (e.g., sorbitol and mannitol), carbohydrates (e.g. lactose), propylene glycol alginate, gellan gum, guar, pectin, tragacanth gum, gum acacia, locust bean gum, gum arabic, gelatin, as well as mixtures of these thickeners. These thickeners are typically included in the compositions of the present invention at levels up to about 0.1%, depending on the particular thickener involved and the viscosity effects desired.

The solid and liquid (food and beverage) compositions of the present invention can, and typically will, contain an effective amount of one or more sweeteners, including carbohydrate sweeteners and natural and/or artificial no/low calorie sweeteners. The amount of the sweetener used in the compositions of the present invention will vary, but typically depends on the type of sweetener used and the sweetness intensity desired.

The compositions of the present invention, regardless of the mode of administration, may also contain various known and conventional cosmetic adjuvants so long as they do not detrimentally affect the desired increase in synthesis of pro-collagen and/or collagen provided by the composition. For example, a composition of the present invention can further include one or more additives or other optional ingredients well known in the art, which can include but are not limited to fillers (e.g., solid, semi-solid, liquid, etc.); carriers; diluents; thickening agents (e.g. xanthan gum); gelling agents; emulsifying agents (e.g., lecithin or acacia); occlusive agents (e.g. caprylic or capric triglycerides); vitamins, retinoids, and retinols (e.g., vitamin B₃, vitamin A, etc.); pigments; fragrances; sunscreens and sunblocks; anti-oxidants and radical scavengers; organic hydroxy acids; exfoliants; skin conditioners; moisturizers (e.g. stearic acid, cetyl alcohol, behenyl alcohol); ceramides, pseudoceramides, phospholipids, sphingolipids, cholesterol, glucosamine, pharmaceutically acceptable penetrating agents (e.g., n-decylmethyl sulfoxide, lecithin organogels, tyrosine, lysine, etc.); preservatives (e.g. methylparaben, chlorphenesin); antimicrobial agents; amino acids such as proline, pyrrolidone carboxylic acid, its derivatives and salts, saccharide isomerate, panthenol, buffers together with a base such as triethanolamine or sodium hydroxide; waxes, such as beeswax, ozokerite wax, paraffin wax; plant extracts, such as Aloe Vera, cornflower, witch hazel, elderflower, or cucumber and combinations thereof. Other suitable additives and/or adjuncts are described in U.S. Pat. No. 6,184,247, the entire contents of which are incorporated herein by reference.

The composition can include additional inactive ingredients, including, but not limited to surfactants, co-solvents, and excipients. Surfactants, such as hydrophilic and hydrophobic surfactants, can be included in the compositions. Particular surfactants can be used based on the on the overall composition of the composition and the intended delivery of the composition. Useful surfactants include polyethoxylated (PEG) fatty acids, PEG-fatty acid diesters, PEG-fatty acid mono- and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, polysaccharide esters, polyethylene glycol alkyl phenols, polyoxyethylene-polyoxypropylene block copolymers, sorbitan fatty acid esters, lower alcohol fatty acid esters, ionic surfactants, and mixtures thereof.

The compositions can also include co-solvents such as alcohols and polyols, polyethylene glycols, ethers, amides, esters, other suitable co-solvents, and mixtures thereof. The compositions can also include excipients or additives such as sweeteners, flavorants, colorants, antioxidants, preservatives, chelating agents, viscomodulators, tonicifiers, odorants, opacifiers, suspending agents, binders, and mixtures thereof.

Other additives that may be included in compositions of the present invention will be apparent to those of skill in the art and are included within the scope of the present invention.

Allantoin is known to be easily absorbed through the skin. Thus, as discussed above, in one example, compositions of the present invention are delivered topically. In other examples, as discussed above, compositions of the present invention may be delivered orally or parenterally. Generally, the compositions of the present invention are administered at least on a daily basis. Administration of the compositions of the invention may continue for any suitable period of time. It should be appreciated that the degree of increase in pro-collagen and/or collagen synthesis and therefore the degree of cosmetic enhancement such as improvement in skin appearance, firmness, or texture will vary directly with the total amount and frequency of composition used.

In one example, a composition of the present invention is topically administered at least once a day. In another example, a composition of the present invention may be administered twice daily. In a further example, a composition of the present invention may be administered three to five times daily. In another example, there is no limit on the amount of a composition of the present invention that might be administered daily. For best effect, compositions of the present invention are administered on at least a daily basis for at least a week to several weeks. Compositions of the present invention also may be administered on at least a daily basis for several weeks to a month to several months to a year to years. It should be appreciated that there is no limit on how frequently or how long the composition of the present invention is administered.

It is intended that the foregoing detailed description be regarded as illustrative rather than limiting. The present invention is further illustrated by the following experimental investigations and examples, which should not be construed as limiting. The contents of all references, patents and published applications cited throughout this patent are hereby incorporated by reference herein.

### EXAMPLES

### Example 1

### Pro-Collagen Synthesis In Vitro Assay

Hs27/HEK001 cells are plated at 10,000/40,000 cells per well of 96-well plate. Hs27 cells are normal human fibroblasts derived from a foreskin source. HEK100 cells are a human epidermal cell line that originally was derived from human scalp keratinocytes. Outside wells of the 96-well plate do not contain any cells and should contain buffer alone or media alone to improve heating and minimize edge effects. Hs27/HEK001 cells are allowed to attach overnight. Following overnight incubation, media is removed from the wells and replaced with 100 μl fresh media.

Stock solutions of an allantoin sample are prepared (50 mg/ml) in DMSO:ethanol:dlH₂O at concentrations of 5:3:2. After overnight incubation, the test samples are added to wells in triplicate to final concentrations of 1, 10, and 100 μg/ml. The cells with test sample added are incubated overnight and supernatants are collected for analysis using a Procollagen ELISA kit from Takara Bio, Inc. (Japan). The EC50 concentration for each test sample is monitored and recorded to evaluate variability over time.

Controls for this assay include administration of 0.1, 1, and 10 μg/ml ascorbic acid and all-trans-retinoic acid to H27/HEK cells. Untreated cells also are used as a negative control. It generally is known that ascorbic acid increases pro-collagen synthesis by approximately 15-20% compared to untreated control.

Each sample/control concentration in the assay was run in triplicate wells. Allantoin exhibited a dose-dependent increase in pro-collagen synthesis with increasing amounts of allantoin. The average of the results achieved administering allantoin at 100 μg/ml is reported below in Table I. Administration of allantoin in this example resulted in a significant increase in pre-collagen production as measured using the pro-collagen ELISA kit from Takara Bio, Inc. (Japan). Specifically, at 100 μg/ml, allantoin resulted in an average increase of 24.1% in pro-collagen synthesis. Increases also were seen for glycosaminoglycan production, an average increase of 11.2%, and for elastin, an average increase of 6.2%.

TABLE I
Effect of Allantoin Administration (at 100 μg/ml)

| | Percent Increase vs. |
|---|---|
| Effect of Allantoin Administration on: | Untreated Control: |
| Pro-Collagen synthesis | 124.1% |
| Synthesis of glycosaminoglycans | 111.2% |
| Elastin synthesis | 106.2% |
| Hyaluronic acid synthesis | 79.7% |

### Example 2

### Cellular Viability

Fresh media is added to the wells of Example 1 following collection of the supernatants along with 10 μl WST-1 reagent to assess viability. The fresh media and WST-1 reagent are incubated with the cells for approximately 1 hour. The well-plate is then read at 450 nm to assess cellular viability.

### Example 3

### In Vivo Anti-Wrinkle Evaluation Study

A formulation of the present invention was evaluated in 25 female subjects with ages ranging from 35-50. The selected subjects possessed periorbital wrinkles, e.g. “crow's feet,” which were confirmed by a trained dermatologist's medical interview and physical examination. Subjects meeting any of the following criteria were excluded from participation in the study: dermatologic disease on the test site, infectious skin disease, atopic dermatitis, use of anti-wrinkle functional cosmetics with retinol or fruit acid in the last 3 months, history of skin scaling and skin care in the last 3 months, history of chemical peel or wrinkle elimination in the last 6 months, exhibit hypersensitivity and allergy to cosmetics, shampoo, medicines, exposure to sunlight, pregnant or lactating, malnutrition, or drug abusers or alcoholics. Subjects were prohibited from using any other skin care products or methods including eye creams, whitening products, anti-wrinkle product, packs, massages, etc. for one week prior to the study and for the duration of the study.

Subjects were randomly divided into two groups: Group A and Group B. In Group A subjects, the formulation of the present invention was tested by application to the left periorbital area. Group A subjects were given a placebo composition to apply to the right periorbital area. In Group B subjects, the formulation of the present invention was tested by application to the left periorbital area. Group B subjects were given a placebo composition to apply to the left periorbital area.

The formulation of the present invention tested in this example comprised approximately 0.15% allantoin along with other ingredients including water, emollients, emulsifiers, humectants, occlusive agents, moisturizers, thickeners, and preservatives. More specifically, the formulation comprised approximately 0.15% allantoin and water as the base carrier. Additional ingredients in the test formulation included isononyl isononanoate, hydrogenated lecithin, butylene glycol, glycerin, caprylic/capric triglyceride, stearic acid, cetyl alcohol, behenyl alcohol, xanthan gum, methylparaben, and chlorphenesin.

The study lasted 12 weeks. At four points during the study, skin topography in the periorbital areas of each subject was scored visually and mechanically. For visual assessment, a double blind test was used and each subject's periorbital wrinkles were evaluated by 2 dermatologists trained in visual assessment of photodamage to skin. The first visual assessment occurred before any application of the test formulation (week 0). Each subject's periobital wrinkles were again evaluated by the same 2 dermatologists at weeks 4, 8 and 12. The dermatologists used the global photodamage score (0: none, 1: none/mild, 2: mild, 3: mild/moderate, 4: moderate, 5: moderate/severe, 6: severe, 7: very severe, see Arch Dermatol vol. 137(8):1043-1051, 2001) to evaluate each subject's periorbital wrinkles. In addition, at each evaluation, the dermatologists photographed the periorbital areas for each subject (using a digital camera, OLYMPUS E-1, JAPAN).

Visual assessment by the trained dermatologists was scored so that the differences between the test product group and the control group were compared using an independent t-test or paired t-test (significance sets at p<0.05). Results of this study are reported below at Table 2.

TABLE 2
Visual Assessment
Visual Photodamage Score

| | Week 0 | Week 4 | Week 8 | Week 12 |
|---|---|---|---|---|
| Placebo | 4.04 | 4.08 | 4.00 | 3.96 |
| Allantoin | 4.13 | 4.04 | 3.96 | 3.75 |

The visual assessment revealed that the test formulation reduced the visual appearance of the number of wrinkles and also visually reduced the appearance of the depth of the wrinkles. This result was significant compared to placebo.

At each evaluation, the determatologists also performed a mechanical assessment of each subject. Replicas of the periorbital areas for each subject were created, and evaluated by using a Visiometer (Skin-Visiometer SV 600, Courage & Khazaka, Germany). Light intensity is analyzed with Lambert & Beer's Law, and the degree of skin wrinkle improvement is measured. (Iₑₓ=Iᵢₙ e^{-kd})

For Visiometer measurements, the differences between the two groups (test and placebo) were compared using an independent t-test or paired t-test (significance sets at p<0.05). Results for this test are reported below at Tables 3 and 4. Table 3 reports the average roughness observed for each subject at the end of the twelve week period while Table 4 reports the maximum roughness observed for each subject.

TABLE 3
Average Roughness - 12 Week Results

| | Calculated fields | | | | | R3 |
|---|---|---|---|---|---|---|
| | Blank | | | R3 | | With |
| | Baseline - | With Allantoin | R3 | With | R3 | Allantoin |
| Subject | 12 week | Baseline - 12 | Blank | Allantoin | Blank 12 | 12 |
| No. | results | week results | Baseline | Baseline | weeks | weeks |
| 1 | 0.04 | 0.03 | 0.2 | 0.19 | 0.16 | 0.16 |
| 2 | −21.71 | 0.06 | 0.29 | 0.26 | 22 | 0.2 |
| 3 | −0.01 | 0.01 | 0.22 | 0.24 | 0.23 | 0.23 |
| 4 | 0.06 | 0.06 | 0.27 | 0.27 | 0.21 | 0.21 |
| 5 | 0.01 | 0.05 | 0.19 | 0.23 | 0.18 | 0.18 |
| 6 | 0 | 0.02 | 0.22 | 0.22 | 0.22 | 0.2 |
| 7 | 0.04 | 0.01 | 0.22 | 0.18 | 0.18 | 0.17 |
| 8 | 0.01 | 0.04 | 0.22 | 0.22 | 0.21 | 0.18 |
| 9 | 0.02 | 0.03 | 0.22 | 0.21 | 0.2 | 0.18 |
| 10 | 0.01 | 0.03 | 0.25 | 0.28 | 0.24 | 0.25 |
| 11 | 0.03 | 0.05 | 0.25 | 0.27 | 0.22 | 0.22 |
| 12 | 0.01 | −0.01 | 0.25 | 0.24 | 0.24 | 0.25 |
| 14 | 0.06 | 0.05 | 0.27 | 0.25 | 0.21 | 0.2 |
| 15 | 0.04 | 0.05 | 0.23 | 0.22 | 0.19 | 0.17 |
| 16 | 0 | 0.03 | 0.2 | 0.22 | 0.2 | 0.19 |
| 17 | 0.01 | 0.01 | 0.22 | 0.22 | 0.21 | 0.21 |
| 18 | 0.03 | 0.06 | 0.2 | 0.21 | 0.17 | 0.15 |
| 19 | 0.05 | 0.05 | 0.23 | 0.23 | 0.18 | 0.18 |
| 20 | 0.06 | 0.03 | 0.24 | 0.22 | 0.18 | 0.19 |
| 21 | 0.05 | 0.09 | 0.23 | 0.27 | 0.18 | 0.18 |
| 22 | 0.04 | 0.02 | 0.19 | 0.17 | 0.15 | 0.15 |
| 23 | 0.04 | 0.05 | 0.24 | 0.23 | 0.2 | 0.18 |
| 24 | 0.07 | 0.1 | 0.23 | 0.27 | 0.16 | 0.17 |
| 25 | 0.05 | 0.07 | 0.24 | 0.22 | 0.19 | 0.15 |
| Avg | | | 0.232 | 0.232 | 0.200 | 0.190 |
| STD | | | 0.026 | 0.029 | 0.020 | 0.030 |

TABLE 4
Maximum roughness - 12 Week Results

| | Calculated fields | | | | | R2 |
|---|---|---|---|---|---|---|
| | Blank | | | R2 | | With |
| | Baseline - | With Allantoin | R2 | With | R2 | Allantoin |
| Subject | 12 week | Baseline - 12 | Blank | Allantoin | Blank 12 | 12 |
| No. | results | week results | Baseline | Baseline | weeks | weeks |
| 1 | 0.06 | 0.09 | 0.29 | 0.32 | 0.23 | 0.23 |
| 2 | 0.11 | 0.16 | 0.42 | 0.42 | 0.31 | 0.26 |
| 3 | −0.02 | 0.01 | 0.31 | 0.35 | 0.33 | 0.34 |
| 4 | 0.09 | 0.09 | 0.38 | 0.38 | 0.29 | 0.29 |
| 5 | 0.01 | 0.07 | 0.27 | 0.34 | 0.26 | 0.27 |
| 6 | 0.03 | 0.05 | 0.32 | 0.33 | 0.29 | 0.28 |
| 7 | 0.17 | 0.02 | 0.41 | 0.26 | 0.24 | 0.24 |
| 8 | 0.01 | 0.04 | 0.33 | 0.31 | 0.32 | 0.27 |
| 9 | 0.09 | 0.04 | 0.35 | 0.28 | 0.26 | 0.24 |
| 10 | 0.02 | 0.06 | 0.36 | 0.41 | 0.34 | 0.35 |
| 11 | 0.05 | 0.09 | 0.37 | 0.4 | 0.32 | 0.31 |
| 12 | 0.01 | 0.03 | 0.36 | 0.38 | 0.35 | 0.35 |
| 14 | 0.1 | 0.09 | 0.4 | 0.36 | 0.3 | 0.27 |
| 15 | 0.01 | 0.17 | 0.35 | 0.41 | 0.34 | 0.24 |
| 16 | 0 | 0.04 | 0.29 | 0.3 | 0.29 | 0.26 |
| 17 | 0.05 | 0.05 | 0.34 | 0.34 | 0.29 | 0.29 |
| 18 | 0 | 0.11 | 0.27 | 0.33 | 0.27 | 0.22 |
| 19 | 0.05 | 0.15 | 0.33 | 0.41 | 0.28 | 0.26 |
| 20 | 0.12 | 0.09 | 0.4 | 0.35 | 0.28 | 0.26 |
| 21 | 0.07 | 0.16 | 0.34 | 0.39 | 0.27 | 0.23 |
| 22 | 0.03 | 0.04 | 0.25 | 0.25 | 0.22 | 0.21 |
| 23 | 0.07 | 0.13 | 0.33 | 0.37 | 0.26 | 0.24 |
| 24 | 0.13 | 0.36 | 0.36 | 0.6 | 0.23 | 0.24 |
| 25 | 0.08 | 0.18 | 0.37 | 0.39 | 0.29 | 0.21 |
| Avg | | | 0.343 | 0.361 | 0.290 | 0.270 |
| STD | | | 0.045 | 0.068 | 0.040 | 0.040 |

The results reported at Tables 3 and 4 demonstrate that the test formulation achieved a significant reduction in the depth of wrinkles over time. An increase in pro-collagen or collagen synthesis reduces the depth and appearance of wrinkles.

The above descriptions are those of the preferred embodiments of the invention. Various alterations and changes can be made without departing from the spirit and broader aspects of the invention as defined in the appended claims, which are to be interpreted in accordance with the principles of patent law including the doctrine of equivalents. Any references to claim elements in the singular, for example, using the articles “a,” “an,” “the,” or “said,” is not to be construed as limiting the element to the singular.

1. A method of increasing pro-collagen synthesis in mammalian skin comprising administering a composition comprising allantoin.

2. The method of claim 1, wherein the mammalian skin is human skin.

3. The method of claim 2, wherein the allantoin forms approximately 0.05% to approximately 2% by weight of the composition.

4. The method of claim 3, wherein the allantoin forms approximately 0.075% to approximately 1.5% by weight of the composition.

5. The method of claim 4, wherein the allantoin forms approximately 0.1% to approximately 1% by weight of the composition.

6. The method of claim 5, wherein the allantoin forms approximately 0.25% to approximately 0.75% by weight of the composition.

7. The method of claim 6, wherein the allantoin forms approximately 0.5% by weight of the composition.

8. The method of claim 2, wherein the composition is topically administered.

9. The method of claim 3, wherein the composition is topically administered as a cream, lotion, gel, paste, spray, tonic, or emulsion.

10. The method of claim 1, wherein the composition further comprises one or more of penetration enhancers, moisturizers, fragrances, thickening agents, gelling agents, vitamins, retinoids, and retinols, pigments, exfoliants, antioxidants, skin conditioners.

11. The method of claim 2, wherein the composition is orally administered.

12. The method of claim 11, wherein the composition is orally administered in the form of a pill, tablet, capsule, or liquid.

13. A method of decreasing the appearance and depth of wrinkles and fine lines in mammalian skin comprising administering a composition comprising allantoin, wherein administration of allantoin increases pro-collagen synthesis in the mammalian skin.

14. The method of claim 13, wherein the mammalian skin is human skin.

15. The method of claim 14, wherein the allantoin forms approximately 0.05% to approximately 2% by weight of the composition.

16. The method of claim 15, wherein the allantoin forms approximately 0.075% to approximately 1.5% by weight of the composition.

17. The method of claim 16, wherein the allantoin forms approximately 0.1% to approximately 1% by weight of the composition.

18. The method of claim 17, wherein the allantoin forms approximately 0.25% to approximately 0.75% by weight of the composition.

19. The method of claim 18, wherein the allantoin forms approximately 0.5% by weight of the composition.

20. The method of claim 13, wherein the composition is topically administered.

21. The method of claim 20, wherein the composition is topically administered as a cream, lotion, gel, paste, spray, tonic, or emulsion.

22. The method of claim 13, wherein the composition further comprises one or more of penetration enhancers, moisturizers, fragrances, thickening agents, gelling agents, vitamins, retinoids, and retinols, pigments, exfoliants, antioxidants, skin conditioners.

23. The method of claim 13, wherein the composition is orally administered.

24. The method of claim 23, wherein the composition is orally administered in the form of a pill, tablet, capsule, or liquid.
